(19) United States
(12) Patent Application Publication
(10) Pub. No.: US 2026/0199197 A1
(43) Pub. Date: Jul. 16, 2026

(54) OIL-IN-WATER EMULSION COSMETIC

(71) Applicant: SHISEIDO COMPANY, LTD., Chuo-ku, Tokyo (JP)

(72) Inventors: Shota MIZUNO, Tokyo (JP); Kayoko NAOI, Tokyo (JP)

(73) Assignee: SHISEIDO COMPANY, LTD., Chuo-ku, Tokyo (JP)

(21) Appl. No.: 19/133,451

(22) PCT Filed: Dec. 14, 2023
(86) PCT No.: PCT/JP2023/044777

§ 371 Date: May 28, 2025

(30) Foreign Application Priority Data
Dec. 28, 2022 (JP) 2022-211812

(51) Int. Cl. A61K 8/06; A61K 8/27; A61K 8/29; A61Q 17/04

(52) U.S. Cl. CPC A61K 8/062; A61K 8/27; A61K 8/29; A61Q 17/04; A61K 2800/612

(57) ABSTRACT

An objective of the present invention is to provide an oil-in-water emulsion cosmetic that stably contains, in an internal oil phase, large-particle zinc oxide having an average particle diameter of 0.5 to 5 μm. The oil-in-water emulsion cosmetic according to the present invention is characterized by comprising (A) zinc oxide having an average particle diameter of 0.5 to 5 μm that has been subjected to a hydrophobization treatment, and (B) hydrophobized fine-particle zinc oxide, wherein the (B) hydrophobized fine-particle zinc oxide content is 5% by mass or more with respect to the overall amount of the cosmetic.

### TECHNICAL FIELD

The present invention relates to an oil-in-water emulsion cosmetic that stably contains, in an internal oil phase, large-particle zinc oxide having an average particle diameter of 0.5 to 5 μm.

### BACKGROUND ART

Protecting the skin from harm due to ultraviolet rays is one of the important problems in skin care and body care, and various UV care cosmetics for minimizing the detrimental impact of ultraviolet rays on the skin have been developed. UV care cosmetics generally protect the skin from harm due to ultraviolet rays by covering the skin with a coating film in which an ultraviolet absorbing agent or an ultraviolet scattering agent is blended, thereby absorbing or scattering UVA and UVB and suppressing the amount of ultraviolet radiation reaching the skin (Non-Patent Document 1).

In recent years, sun-care components known as “ultraviolet wavelength conversion substances” or “phosphors” that deal with ultraviolet rays by a mechanism different from ultraviolet absorbing agents and ultraviolet scattering agents have received interest.

Ultraviolet wavelength conversion substances are substances that convert the wavelengths of ultraviolet rays (peak wavelength: 200 nm to 400 nm) contained in input light, and that emit output light (peak wavelength: 500 nm to 700 nm) having wavelengths longer than the wavelengths of the ultraviolet rays. For example, as described in Patent Document 1, various ultraviolet wavelength conversion substances are known, whether they are organic compounds or inorganic compounds, as described in Patent Document 1.

Since ultraviolet wavelength conversion substances absorb and convert the wavelengths of ultraviolet rays to visible light without scattering them, they have the advantage of being able to produce a natural skin color in comparison with ultraviolet scattering agents, such as titanium oxide and zinc oxide, which have been conventionally used. Additionally, ultraviolet wavelength conversion substances also have the function of activating skin cells by making effective use of ultraviolet rays, thus having the effect of preventing and ameliorating wrinkles, blotches, skin aging, photoaging, etc.

Various attempts have been made towards utilizing the above-mentioned characteristic effects of ultraviolet wavelength conversion substances to cosmetics. For example, Patent Document 1 proposes a skin composition containing (A) an ultraviolet wavelength conversion substance, (B) a total content of 10% by weight or more of a hydrocarbon oil and/or a linear silicone oil, and (C) a total powder content of 0% to 65% by weight. Additionally, Patent Document 2 proposes an emulsion composition containing (A) an ultraviolet wavelength conversion substance and (B) an organic oil phase thickener.

However, the zinc oxide that is widely used as an inorganic compound-based ultraviolet wavelength conversion substance (hereinafter referred to as “zinc oxide phosphor” in accordance with Patent Document 1, etc.) has a large average particle diameter of 1 to 4 μm, and therefore is difficult to stably blend into a base and tends to undergo aggregation, precipitation, etc. For this reason, zinc oxide phosphors are generally blended into water-in-oil emulsion cosmetics in which relatively large amounts of oil components can be blended, as in Example 5 in Patent Document 1 and Example 5 in Cited Document 2. However, since water-in-oil emulsion cosmetics have external phases constituted by oil components, when they are applied to skin, it is difficult to realize a refreshing texture in use like that of oil-in-water emulsion cosmetics.

Meanwhile, oil-in-water emulsion cosmetics can obtain a fresh and watery texture when applied to the skin, and are therefore widely used as bases for external preparations for the skin, such as skin cosmetics, that are directly applied to the skin. Since the protection of skin from ultraviolet rays by skin care and body care has become a matter of daily practice, the importance of such UV care cosmetics using oil-in-water emulsion cosmetics as bases has increased.

Patent Document 3 proposes an oil-in-water emulsion cosmetic containing an ultraviolet wavelength conversion substance, and also discloses an example in which a zinc oxide phosphor is used as an ultraviolet wavelength conversion substance (Example 4). However, the blended amount of the zinc oxide phosphor is held somewhat low, and there were also cases in which the stability was insufficient.

### RELATED ART

### Patent Documents

Patent Document 1: JP 2021-172607 A
Patent Document 2: JP 2020/204196 A
Patent Document 3: JP 2020/204189 A

### Non-Patent Documents

Non-Patent Document 1: Shin-keshohingaku, second edition, edited by Takeo Mitsui, 2001, published by Nanzando, pp. 497-504.

### SUMMARY OF THE INVENTION

### Problem to be Solved by the Invention

An objective of the present invention is to provide an oil-in-water emulsion cosmetic that stably contains, in the internal oil phase, large-particle zinc oxide having an average particle diameter of 0.5 to 5 μm.

### Means for Solving the Problem

The present inventors carried out diligent investigations towards solving the above-mentioned problem, as a result of which they discovered that even large-particle zinc oxide can be stably blended into the internal oil phase of an oil-in-water emulsion cosmetic by performing a hydrophobization treatment on the surface thereof and also making combined use of a prescribed amount of hydrophobized fine-particle zinc oxide, thereby completing the present invention.

That is, the present invention provides an oil-in-water emulsion cosmetic comprising:
(A) zinc oxide having an average particle diameter of 0.5 to 5 μm that has been subjected to a hydrophobization treatment (hereinafter sometimes referred to as “hydrophobized zinc oxide”); and
(B) hydrophobized fine-particle zinc oxide,
wherein the (B) hydrophobized fine-particle zinc oxide content is 5% by mass or more with respect to the overall amount of the cosmetic.

### Effects of the Invention

By having the above-mentioned configuration, the present invention can stably blend large-particle zinc oxide, in the form of hydrophobized zinc oxide, in the internal oil phase of an oil-in-water emulsion cosmetic. For this reason, a fresh texture in use characteristic of oil-in-water emulsion cosmetics can be realized.

Additionally, by using a large-particle zinc oxide having an ultraviolet wavelength conversion function, a natural skin color can be produced while providing protection against ultraviolet rays, and a cell activation effect can also be obtained.

### MODES FOR CARRYING OUT THE INVENTION

The oil-in-water emulsion cosmetic of the present invention is characterized by comprising (A) hydrophobized zinc oxide and (B) hydrophobized fine-particle zinc oxide under prescribed conditions. Hereinafter, the respective components constituting the oil-in-water emulsion cosmetic of the present invention will be explained in detail.

### <(A) Hydrophobized Zinc Oxide>

The (A) hydrophobized zinc oxide blended in the present invention is obtained by implementing a hydrophobization treatment on the surface of large-particle zinc oxide having an average particle diameter of 0.5 to 5 μm.

The large-particle zinc oxide constituting the (A) hydrophobized zinc oxide is not particularly limited as long as the average particle diameter is within the range from 0.5 to 5 μm, preferably within the range from 0.7 to 4.5 μm, and more preferably within the range from 1 to 4 μm.

In the present specification, “average particle diameter” refers to the particle diameter at a cumulative value of 50% in a particle size distribution determined by a laser diffraction/scattering method.

Since the average particle diameter of the zinc oxide constituting the (A) hydrophobized zinc oxide is large, being from 0.5 to 5 μm, a highly functional type such as zinc oxide having an ultraviolet wavelength conversion function can be used. Using a zinc oxide having an ultraviolet wavelength conversion function is preferable for being able to impart an ultraviolet protection effect and a cell activation effect to the cosmetic.

As zinc oxides having an ultraviolet wavelength conversion function, zinc oxide doped with a sulfur-containing compound is known, and can be produced by a method described, for example, in WO 2018/004006, JP 2018-131422 A or JP H5-117127 A.

As the hydrophobization treatment for the (A) hydrophobized zinc oxide, there are, for example, silane compound treatments (triethoxycaprylylsilane, etc.), silicone compound treatments, fluorine-modified silicone compound treatments, fluorine compound treatments, higher fatty acid treatments (stearic acid, etc.), higher alcohol treatments, fatty acid ester treatments, metal soap treatments, amino acid treatments, alkyl phosphate treatments, etc. Among the above, stearic acid treatments are particularly preferable.

The blended amount of the (A) hydrophobized zinc oxide should preferably be 0.01% by mass or more, more preferably 1% by mass or more, and should preferably be 10% by mass or less, more preferably 7% by mass or less, with respect to the overall amount of the oil-in-water emulsion cosmetic. Thus, the blended amount range may be from 0.01% to 10% by mass, from 1% to 7% by mass, etc.

### <(B) Hydrophobized Fine-Particle Zinc Oxide>

The (B) hydrophobized fine-particle zinc oxide blended into the present invention is a fine-particle zinc oxide that has conventionally been used as an ultraviolet scattering agent, the surface of which has been hydrophobized.

The (B) hydrophobized fine-particle zinc oxide has an average particle diameter within the range from 2 to 300 nm, preferably within the range from 5 to 100 nm. If the average particle diameter is within this range, the (A) hydrophobized zinc oxide can be stably blended into the internal oil phase of the oil-in-water emulsion cosmetic.

The method for surface treatment of the (B) hydrophobized fine-particle zinc oxide, though not particularly limited, may include silicone compound treatments with methyl hydrogen polysiloxane, methyl polysiloxane, etc.; fluorine compound treatments with a perfluoroalkyl phosphoric acid ester, a perfluoroalcohol, etc.; amino acid treatments with N-acylglutamic acid, etc.; silane compound treatments with triethoxycaprylylsilane, etc.; and aside from the above, lecithin treatments; metal soap treatments; fatty acid treatments; alkyl phosphoric acid treatments, etc. Among the above, silicone compound treatments are particularly preferable.

The silicone compounds that can be used in the silicone compound treatment include, for example, various types of silicone oils such as methyl polysiloxane, methyl phenyl polysiloxane, methyl hydrogen polysiloxane, methyl cyclopolysiloxane, octamethyl cyclotetrasiloxane, decamethyl cyclopentasiloxane, dodecamethyl cyclohexasiloxane, octamethyl trisiloxane, tetradecamethyl hexasiloxane, dimethyl siloxane/methyl (polyoxyethylene) siloxane/methyl (polyoxypropylene) siloxane copolymer, dimethyl siloxane/methyl (polyoxyethylene) siloxane copolymer, dimethyl siloxane/methyl (polyoxyethylene) siloxane copolymer, dimethyl siloxane/methyl (polyoxypropylene) siloxane copolymer, dimethyl siloxane/methyl cetyloxy siloxane copolymer, dimethyl siloxane/methyl stearoxy siloxane copolymer, etc. Preferably, methyl hydrogen polysiloxane or methyl polysiloxane is used.

The blended amount of the (B) hydrophobized fine-particle zinc oxide should preferably be 5% by mass or more, more preferably 7% by mass or more with respect to the overall amount of the oil-in-water emulsion cosmetic. If the blended amount is 5% by mass or more, the (A) hydrophobized zinc oxide can be stably blended into the internal oil phase of the oil-in-water emulsion cosmetic. On the other hand, though the upper limit of the blended amount is not particularly limited, it should preferably be 15% by mass or less because, even if blended in excess, a commensurate increase in stability cannot be expected. Thus, the blended amount range may be from 5% to 15% by mass, from 7% to 15% by mass, etc. with respect to the overall amount of the cosmetic.

### <Optional Blended Components>

In the oil-in-water emulsion cosmetic of the present invention, aside from the above-mentioned components (A) and (B), components that are normally used in oil-in-water emulsion cosmetics can be blended within a range not compromising the effects of the present invention. For example, aside from oil components, aqueous solvents and ultraviolet absorbing agents, surfactants, hydrophilic thickeners, humectants, medicinal agents, anti-oxidants, chelating agents, preservatives, powders, fragrances, colorants, pigments, etc. can be appropriately blended as needed.

The oil components are not particularly limited as long as they are oil components that are blended into oil-in-water emulsion cosmetics, and may be hydrocarbon oils, silicone oils, higher alcohols, ester oils, fatty acids, oils/fats, waxes, etc.

Hydrocarbon oils include, for example, liquid paraffin, isohexadecane, isododecane, ozokerite, squalane, squalene, pristane, paraffin, isoparaffin, ceresin, vaseline, microcrystalline wax, hydrogenated polyisobutene, olefin oligomers, etc.

Silicone oils include dimethyl polysiloxane, methyl phenyl polysiloxane, hexamethyl cyclotrisiloxane, octamethyl cyclotetrasiloxane, decamethyl cyclopentasiloxane, etc.

Higher alcohols include, for example, linear alcohols such as lauryl alcohol, cetyl alcohol, stearyl alcohol, behenyl alcohol, myristyl alcohol, oleyl alcohol and cetostearyl alcohol; branched alcohols such as monostearyl glyceryl ether (batyl alcohol), 2-decyl tetradecynol, lanolin alcohol, cholesterol, phytosterol, hexyl dodecanol, isostearyl alcohol, octyl dodecanol, etc.

Ester oils include, for example, isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, diisotearyl malate, glycerin di-2-heptylundecanoate, trimethylolpropane tri-2-ethylhexanoate, trimethylolpropane triisostearate, pentaerythrityl tetraethylhexanoate, glycerin tri-2-ethylhexanoate, cetyl 2-ethylhexanoate, 2-ethylhexyl palmitate, glycerin trimyristate, glyceride tri-2-heptylundecanoate, castor oil fatty acid methyl ester, oleyl oleate, acetoglyceride, 2-heptylundecyl palmitate, diisobutyl adipate, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate, triethyl citrate, etc.

Fatty acids include, for example, oleic acid, isostearic acid, linolic acid, linoleic acid, etc.

Oils/fats include, for example, Japan wax, cocoa butter, hydrogenated castor oil, avocado oil, camellia oil, macadamia nut oil, corn oil, olive oil, rapeseed oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, Paulownia fargesii oil, Paulownia tomentosa oil, germ oil, triglycerin, glycerin trioctanoate, glycerin triisopalmitate, etc.

Waxes include, for example, carnauba wax, beeswax, candelilla wax, jojoba wax, etc.

Aqueous solvents include, for example, water (purified water, ion-exchanged water, tap water, etc.), lower alcohols, or mixtures thereof.

Ultraviolet absorbing agents include, for example, 2-ethylhexyl para-methoxycinnamate, oxybenzone, 4-t-butyl-4′-methoxydibenzoylmethane, octyl triazone, bis-ethylhexyloxyphenol methoxyphenyl triazine, dioctyl butamido triazone, diethylamino hydroxybenzoyl hexyl benzoate, 2-cyano-3,3-diphenyl acrylic acid 2′-ethylhexyl ester, polysilicone-15, octyl salicylate, homomenthyl salicylate, p-methylbenzylidene camphor, etc.

While fine-particle titanium oxide, which is widely known to be an ultraviolet scattering agent, can also be blended into the oil-in-water emulsion cosmetic according to the present invention, the stability of the cosmetic tends to become worse. Therefore, it is preferable not to blend fine-particle titanium oxide at all, or in the case in which fine-particle titanium oxide is blended, to blend less than 1% by mass with respect to the overall amount of the cosmetic. Although fine-particle titanium oxide is a component that, like the (B) hydrophobized fine-particle zinc oxide, is classified as an ultraviolet scattering agent, it does not have stabilizing effects like those of the (B) hydrophobized fine-particle zinc oxide. The stabilizing effects provided by the (B) hydrophobized fine-particle zinc oxide in the present invention are surprising and absolutely could not be predicted by a person skilled in the art.

### USE

The oil-in-water emulsion cosmetic of the present invention is not particularly limited in terms of the format thereof, and is widely applicable to any format, such as a toner, a milky lotion, a cream, a gel, an essence (serum), a sunscreen cosmetic, etc.

### Examples

The present invention will be explained in further detail below by providing examples. However, the present invention is not limited, in any way, by these examples. Where not particularly noted, the blended amounts are indicated in percentage by mass with respect to the oil-in-water emulsion cosmetic. Before specifically explaining the respective examples, the evaluation methods that were employed will be explained.

### <Stability>

Each cosmetic (sample) was stored for 4 weeks under 50° C. conditions, and the viscosity and pH were measured. The rate of change was determined from the measurement value before storage (initial value) and the measurement value after storage, and the viscosity stability and the pH stability were evaluated according to criteria below.

### <Evaluation Criteria>

A: Rate of change from initial value lower than 150%
B: Rate of change from initial value 150% or higher and lower than 500%
C: Rate of change from initial value 500% or higher

The oil-in-water emulsion cosmetics of Reference Example 1, Examples 1 to 3, and Comparative Examples 1 to 3 in Table 1 below were prepared, and evaluations were performed based on the above-mentioned evaluation method and evaluation criteria. The evaluation results are also indicated.

TABLE 1

| | Ref. | Comp. | Comp. | Ex. | Ex. | Ex. | Comp. |
|---|---|---|---|---|---|---|---|
| | Ex. 1 | Ex. 1 | Ex. 2 | 1 | 2 | 3 | Ex. 3 |
| Water | bal | bal | bal | bal | bal | bal | bal |
| Ethanol | 7 | 7 | 7 | 7 | 7 | 7 | 7 |
| Glycerin | 2 | 2 | 2 | 2 | 2 | 2 | 2 |
| 1,3-Butylene glycol | 3 | 3 | 3 | 3 | 4 | 3 | 3 |
| PEG-8 | — | — | — | — | 1 | — | — |
| Succinoglycan | 0.2 | 0.2 | 0.2 | 0.2 | 0.2 | 0.2 | 0.2 |
| (Dimethylacrylamide/sodium | 0.65 | 0.65 | 0.65 | 0.65 | 0.4 | 0.65 | 0.65 |
| acryloyldimethyltaurate) crosspolymer | | | | | | | |
| PEG-12 dimethicone | 3 | 3 | — | — | — | — | — |
| PEG/PPG-19/19 dimethicone | — | — | 3 | 2 | 2 | 2 | 2 |
| PEG-40 hydrogenated castor oil | 0.5 | 0.5 | 0.5 | 0.5 | 0.5 | 0.5 | 0.5 |
| Polyhydroxystearic acid | 0.5 | 0.5 | 0.5 | 0.5 | 0.5 | 0.5 | 0.5 |
| Dimethicone | 4 | 4 | 4 | — | — | — | — |
| Isopropyl myristate | 5 | 5 | 5 | 2 | 2 | 2 | 2 |
| Myristyl myristate | — | — | — | 1 | 1 | 1 | 1 |
| Alkyl (C12-15) benzoate | — | — | — | 2 | 2 | 2 | 2 |
| PPG-17 | 1 | 1 | 1 | 1 | 1 | 1 | 1 |
| Ethylhexyl methoxycinnamate | 6 | 6 | 6 | 6 | 6 | 6 | 6 |
| bis-Ethylhexyloxyphenol methoxyphenyl triazine | 2 | 2 | 2 | 2 | 2 | 2 | 2 |
| Diethylamino hydroxybenzoyl hexyl benzoate | 2 | 2 | 2 | 2 | 2 | 2 | 2 |
| Stearic acid-treated zinc oxide (average particle | — | 5.1 | 5.1 | 5.1 | 3.1 | 5.1 | 5.1 |
| diameter 3 μm) | | | | | | | |
| Dimethicone-treated fine-particle zinc oxide | — | — | — | 9 | 9 | 8 | 2 |
| (average particle diameter 20 nm) | | | | | | | |
| Hydrogen dimethicone-treated fine-particle | — | — | 5 | — | — | 0.5 | 1 |
| titanium oxide (average particle diameter 15 nm) | | | | | | | |
| Isostearic acid-treated fine-particle titanium oxide | 5 | 5 | — | — | — | — | — |
| (average particle diameter 30 nm) | | | | | | | |
| Hydrogen dimethicone-treated pigment-grade | — | — | — | — | 0.02 | — | — |
| iron oxide | | | | | | | |
| Sodium pyrosulfite | 0.01 | 0.01 | 0.01 | 0.01 | 0.01 | 0.01 | 0.01 |
| Tocopherol | 0.05 | 0.05 | 0.05 | 0.05 | 0.05 | 0.05 | 0.05 |
| BHT | 0.05 | 0.05 | 0.05 | 0.05 | 0.05 | 0.05 | 0.05 |
| EDTA-2Na | 0.01 | 0.01 | 0.01 | 0.01 | 0.01 | 0.01 | 0.01 |
| Phenoxyethanol | 0.5 | 0.5 | 0.5 | 0.5 | 0.5 | 0.5 | 0.5 |
| Citric acid | 0.05 | 0.05 | 0.05 | 0.05 | 0.05 | 0.05 | 0.05 |
| Tranexamic acid | 2 | 2 | 2 | 2 | 2 | 2 | 2 |
| Total | 100 | 100 | 100 | 100 | 100 | 100 | 100 |
| Viscosity stability | A | C | C | A | A | B | C |
| pH stability | A | C | C | A | A | A | C |

As indicated in Table 1, when (A) hydrophobized zinc oxide was not blended, there was no problem in terms of the viscosity stability and the pH stability of the cosmetic, even when fine-particle titanium oxide was included (Reference Example 1). However, when (A) hydrophobized zinc oxide and fine-particle titanium oxide coexisted, the results were unsatisfactory in terms of viscosity stability and pH stability (Comparative Examples 1 and 2).

In contrast therewith, when (B) hydrophobized fine-particle zinc oxide was blended instead of fine-particle titanium oxide, very excellent viscosity stability and pH stability were able to be achieved (Examples 1 and 2).

Meanwhile, when a small amount (0.5% by mass) of fine-particle titanium oxide was blended in the presence of (B) hydrophobized fine-particle zinc oxide, the viscosity stability and the pH stability were not significantly compromised, though there was a slight decrease in viscosity stability (Example 3). However, when the blended amount of fine-particle titanium oxide was increased to 1% by mass, the viscosity stability and the pH stability were observed to become unsatisfactory (Comparative Example 3).

1. An oil-in-water emulsion cosmetic comprising:
(A) zinc oxide having an average particle diameter of 0.5 to 5 μm that has been subjected to a hydrophobization treatment; and
(B) hydrophobized fine-particle zinc oxide;
wherein the (B) hydrophobized fine-particle zinc oxide content is 5% by mass or more with respect to the overall amount of the cosmetic.

2. The oil-in-water emulsion cosmetic according to claim 1, wherein the (A) hydrophobized zinc oxide is a stearic acid-treated zinc oxide.

3. The oil-in-water emulsion cosmetic according to claim 1, wherein the average particle diameter of the (B) hydrophobized fine-particle zinc oxide is within the range from 2 to 300 nm.

4. The oil-in-water emulsion cosmetic according to claim 1, wherein the (B) hydrophobized fine-particle zinc oxide content is within the range from 5% to 15% by mass with respect to the overall amount of the cosmetic.

5. The oil-in-water emulsion cosmetic according to claim 1, wherein, when the cosmetic contains fine-particle titanium oxide, the fine-particle titanium oxide content is less than 1% by mass with respect to the overall amount of the cosmetic.
